(19) United States
(12) Patent Application Publication
(10) Pub. No.: US 2025/0041520 A1
(43) Pub. Date: Feb. 6, 2025

(54) INJECTION DEVICE AND METHOD FOR USING SAME

(71) Applicant: Hangzhou Qiantang Longyue Biotechnology Co., LTD., Hangzhou (CN)

(72) Inventors: Liqun WU, Hangzhou (CN); Wenbin SUN, Hangzhou (CN); Chenguang WANG, Hangzhou (CN); Shijie WU, Hangzhou (CN)

(21) Appl. No.: 18/925,762

(22) Filed: Oct. 24, 2024

Related U.S. Application Data
(63) Continuation-in-part of application No. PCT/CN2022/131170, filed on Nov. 10, 2022.

(30) Foreign Application Priority Data
May 30, 2022 (CN) 202210596758.0

(51) Int. Cl. A61M 5/20; A61M 5/315

(52) U.S. Cl. CPC A61M 5/20; A61M 5/31501; A61M 5/31513

(57) ABSTRACT

Provided are an injection device, an injection device without changing an original structure of a syringe, and a method for using the same. The injection device includes a barrel (1) with a flange (8) and a push rod (2) slidably connected in the barrel (1). One end of the push rod (2) is provided with a piston (3), and the other end is provided with a thumb rest (4). The push rod (2) or the barrel (1) is detachably connected with a stopping member (5), and the stopping member (5) is provided with a first stopping portion (51) and a second stopping portion (52) at different positions. A distance between the first stopping portion (51) and the second stopping portion (52) is a fixed distance. The push rod (2) is moved, and when the thumb rest (4) or the flange (8) abuts against the first stopping portion (51), the injection device is in an injection start position; and when the thumb rest (4) or the flange (8) abuts against the second stopping portion (52), the injection ends, and a gap is left between the piston (3) and the barrel (1). As long as the fixed distance between the first stopping portion and the second stopping portion is determined, the number of tolerances can be greatly reduced, thereby improving the injection accuracy. The piston (3) and a bottom of the barrel (1) do not squeeze each other, so that the piston will not deform. Therefore, a residual medication in a tapper cavity will not be squeezed out for injection, thereby improving the injection accuracy.

### CROSS-REFERENCE TO RELATED APPLICATIONS

This application is a continuation-in-part application of PCT application No. PCT/CN2022/131170 filed on Nov. 10, 2022, which claims the priority of Chinese prior application No. 2022105967580.0 filed on May 30, 2022.

### BACKGROUND OF THE INVENTION

### Field of the Invention

The disclosure belongs to the technical field of medical syringes, and in particular to an injection device, an injection device without changing an original structure of a syringe, and a method for using the same.

### Description of the Related Art

In medical clinical sites, a syringe with graduation marks is usually used for medication preparation or injection. A typical method for using this syringe is to push/pull a rubber stop in the syringe to make the front edge of the rubber stop aligned with the required graduation mark outside the syringe, so as to set the required dose (in mL). The error of the volumetric graduation marks of the syringe comes from the accumulation of manufacturing tolerances of dimensions of components, graduation printing, etc. The error range is usually 0.2 mL or more. However, this syringe still meets the requirements of relevant regulations and various standards and can meet general clinical needs.

However, insulin and vaccines need to be microinjected, for example, in 50, 100 or 200 microliters, and the error is required to be within ±10%. In this case, the error of 0.1 or 0.2 mL (100 or 200 microliters) of a conventional syringe can no longer meet the requirements of microinjection. Especially in the case of injection of 50 microliters, the error is required to be within ±5 microliters. Moreover, for an ordinary syringe, when the injection dose is determined according to the graduations on the injection barrel, the injection barrel made of glass will refract light, so it is prone to produce deviation when reading the graduation, which will further increase the error.

There have been devices that can meet the requirements of microinjection in the prior art, but all these devices have to realize accurate microinjection by means of various electrical control elements. This makes the devices large in size and inconvenient to carry and use. In addition, there are plenty of components, which makes the manufacturing and assembling process complicated. Moreover, the production cost of the device is very high.

There are some other microsyringes that are simple in structure (as shown in FIG. 1). In the figure, D is the inner diameter of the injection barrel; H is the height of the medication; L1 is the effective length of the injection barrel; L2 is the length of the rubber stop; L3 is the effective length of the push rod; and L4 is the height of the stop block. According to D, the cross-sectional area S of the injection barrel may be calculated, the value of H is calculated according to H=L1+L4−L3−L2, and S is multiplied by H to obtain the dose of microinjection. However, during this process, D, L1, L2, L3 and L4 all have tolerances, and the accumulation of these five tolerances makes an error in dose of about 18%. In addition, when the injection is completed, the rubber stop will squeeze the bottom of the injection barrel, which makes the rubber stop prone to deformation, so that the medication in the tapper cavity will be squeezed out for injection. This will further increase the error of the dose of the medication to about 20% and even more, so that the error will be far beyond the requirement of microinjection.

Moreover, many microsyringes cannot realize microinjection before changing the original syringe structure. In this case, a specific syringe is required during microinjection, and an ordinary syringe is not available, which greatly increases the cost of microinjection.

### BRIEF SUMMARY OF THE INVENTION

In order to solve the technical problem that ordinary syringes in the prior art cannot meet the requirements of microinjection and have excessive errors, the disclosure provides an injection device without changing an original structure of a syringe and a method for using the same that can meet the requirements of microinjection and has small errors.

In view of the defects in the prior art, a first objective of the disclosure is to provide an injection device without changing an original structure of a syringe and a method for using the same that can meet the requirements of microinjection and have small errors, which can solve the technical problem that ordinary syringes in the prior art cannot meet the requirements of microinjection and have excessive errors.

In view of the defects in the prior art, a second objective of the disclosure is to provide an injection device without changing an original structure of a syringe and a method for using the same. In the injection device, a push rod or a barrel is connected with a stopping member, and the stopping member is provided with a first stopping portion and a second stopping portion at different positions; the first stopping portion and the second stopping portion are distributed along an axis direction of the push rod; and a distance between the first stopping portion and the second stopping portion is a fixed distance a.

In order to achieve one of the above objectives, a first technical solution of the disclosure is as follows:

An injection device without changing an original structure of a syringe includes a barrel with a flange and a push rod slidably connected in the barrel. One end of the push rod is provided with a piston, and the other end is provided with a thumb rest. The piston is located inside the barrel, and the thumb rest is located outside the barrel. The push rod or the barrel is connected with a stopping member, and the stopping member is provided with a first stopping portion and a second stopping portion at different positions. The first stopping portion and the second stopping portion are distributed along an axis direction of the push rod. A distance between the first stopping portion and the second stopping portion is a fixed distance a. The fixed distance a is a height H of a medication in the prior art. An inner diameter of the barrel may be obtained from the international standard ISO11040, that is, D in the prior art. According to D, a cross-sectional area S of the barrel may be calculated, and then S is multiplied by a to obtain a dose that needs to be microinjected. In the whole process, only tolerances of D and a are needed, so the medication injection accuracy is greatly improved as compared with the prior art where there are five tolerances. In addition, since the value of D is specified in the international standard ISO11040, as long as the value of a is determined in advance according to actual needs, various requirements of microinjection can be met. Even in the case of 50, 100 or 200 microliters, accurate quantitative injection can be realized.

The push rod is moved, and when the thumb rest or the flange abuts against the first stopping portion, the injection device is in an injection start position; and
when the thumb rest or the flange abuts against the second stopping portion, the injection ends, and a gap is left between the piston and a bottom of the barrel.

The first stopping portion and the second stopping portion do not have a fixed structure, as long as they can play a corresponding stopping role.

The start position and the end position of the injection device during injection are more random and free, and there is no need to start and end the injection according to graduation marks. After the injection ends, a gap is left between the piston and the bottom of the barrel, so that the piston will not squeeze the bottom of the barrel, and the piston will not deform. Therefore, the residual medication in a tapper cavity will not be squeezed out for injection, which further reduces the microinjection error and improves the microinjection accuracy.

In addition, according to the injection device, the stopping member is connected with the push rod or the barrel to add the first stopping portion and the second stopping portion, and the thumb rest or the flange in the original structure of the syringe is fitted therewith to realize the function of accurate quantification. In this way, when fitted with a stopping member, an ordinary syringe is also able to realize the function of accurate quantification without changing its structure, so as to meet the requirements of microinjection, thereby greatly reducing the microinjection cost.

In order to achieve one of the above objectives, a second technical solution of the disclosure is as follows:

An injection device includes a barrel with a flange and a push rod slidably connected in the barrel. One end of the push rod is provided with a piston, and the other end is provided with a thumb rest. The piston is located inside the barrel, and the thumb rest is located outside the barrel. The push rod or the barrel is connected with a stopping member, and the stopping member is provided with a first stopping portion and a second stopping portion at different positions. The first stopping portion and the second stopping portion are distributed along an axis direction of the push rod. A distance between the first stopping portion and the second stopping portion is a fixed distance a. The push rod is moved, and when the thumb rest or the flange abuts against the first stopping portion, the injection device is in an injection start position; and
when the thumb rest or the flange abuts against the second stopping portion, the injection ends, and a gap is left between the piston and a bottom of the barrel.

The first stopping portion and the second stopping portion do not have a fixed structure, as long as they can play a corresponding stopping role.

The start position and the end position of the injection device of the disclosure during injection are more random and free, and there is no need to start and end the injection according to graduation marks. After the injection ends, a gap is left between the piston and the bottom of the barrel, so that the piston will not squeeze the bottom of the barrel, and the piston will not deform. Therefore, the residual medication in a tapper cavity will not be squeezed out for injection, which further reduces the microinjection error and improves the microinjection accuracy.

On the one hand, the disclosure provides an injection device without changing an original structure of a syringe, including a barrel with a flange and a push rod slidably connected in the barrel. The push rod or the barrel is connected with a stopping member, and the stopping member can control a displacement distance of the push rod in the barrel, thereby controlling an injection dose.

By adding a stopping block on the injection device, the stopping block can control the displacement distance of the push rod by controlling the moving distance of the push rod, for example, by stopping the sliding of the push rod in the barrel, so as to control the amount of liquid in the barrel injected outward by the push rod. Since the cross section of the barrel is fixed, as long as the moving distance of the push rod in the barrel is fixed, the amount of liquid pushed out is also fixed, thereby realizing accurate quantification.

The stopping block may be located on the push rod, on the barrel, or between the push rod and the barrel. As long as the stopping block can be fixed and can play a certain stopping role on the push rod when the push rod is pushed, it can control the displacement distance of the push rod in the barrel, thereby controlling the injection dose.

In some embodiments, when the stopping block is located on the push rod, it may be located either on the push rod outside the barrel or on the push rod inside the barrel. As long as the stopping block can limit the sliding distance of the push rod in the barrel, the injection dose can be accurately controlled.

Further, the stopping member is provided with a first stopping portion and a second stopping portion at different positions. A distance between the first stopping portion and the second stopping portion is a fixed distance a. The push rod is moved, and when the push rod is stopped by the first stopping portion, the injection device is in an injection start position; and when the push rod is stopped by the second stopping portion, the injection ends.

Further, the stopping member is located on the push rod outside the barrel.

The stopping member is located on the push rod outside the barrel, so that the stopping member can be directly arranged on the original syringe, which is convenient and controllable.

Further, the barrel is provided with a flange, and one end of the push rod is provided with a thumb rest. When the thumb rest or the flange abuts against the first stopping portion, the injection device is in an injection start position; and when the thumb rest or the flange abuts against the second stopping portion, the injection ends.

According to the injection device of the disclosure, the stopping member is connected with the push rod or the barrel to add the first stopping portion and the second stopping portion, and the thumb rest or the flange in the original structure of the syringe is fitted therewith to realize the function of accurate quantification.

Further, the push rod is clamped by the stopping member. The other end of the push rod is provided with a piston, and a gap is left between the piston and a bottom of the barrel.

The piston is elastic, when the piston contacts the bottom of the barrel and deforms, the quantification process is prone to errors. Therefore, preferably, a gap is left between the piston and the bottom of the barrel, that is, the injection ends before the piston comes into contact with the bottom of the barrel, thereby improving the control accuracy of quantitative injection.

Of course, if the sliding distance of the push rod can be controlled accurately such that the force of the piston coming into contact with the bottom of the barrel every time is consistent, even if the piston contacts the bottom of the barrel, accurate quantification can still be realized, which, however, will increase the control difficulty and is not a preferred method.

In some embodiments, the first stopping portion and the second stopping portion may not be flat, as long as several points protruding from the first stopping portion or the second stopping portion can abut against the thumb rest or the flange to make the push rod stop moving. Of course, the first stopping portion and the second stopping portion are preferably structures matched with the thumb rest or the flange. When the surface of the thumb rest or the flange for abutting is flat, the first stopping portion and the second stopping portion are also preferably flat.

Further, the push rod is clamped by the stopping member, and one end of the stopping member abuts against the thumb rest. The other end of the stopping member extends outward along a radial direction of the push rod to form a first protrusion, and an end of the first protrusion away from the stopping member extends toward the barrel to form a second protrusion. An end surface of the second protrusion facing the barrel is the first stopping portion. An end surface of the first protrusion facing the barrel is the second stopping portion. Compared with electrical elements in the prior art, the stopping member is more simple in structure, which reduces the use cost and makes the whole microinjection operation more simple and more convenient.

When the flange abuts against the first stopping portion, the injection device is in the injection start position.

When the flange abuts against the second stopping portion, the injection ends.

Further, the stopping member includes a first locating block and a second locating block, and the first locating block and the second locating block are connected and separated detachably.

Further, the stopping member includes a first locating block and a second locating block. The first locating block is clamped on the flange, and a top of the first locating block is connected with the second locating block. An end surface of the second locating block facing the thumb rest is the first stopping portion, and an end surface of a top of the first locating block facing the thumb rest is the second stopping portion. The stopping member may be an integrated structure or a split structure. The split structure is convenient for subsequent demounting during microinjection.

When the thumb rest abuts against the first stopping portion, the injection device is in the injection start position.

When the thumb rest abuts against the second stopping portion, the injection ends.

When the first locating block and the second locating block are combined, the injection device is in the injection start position. After the second locating block is disassembled and taken out, the push rod is pushed to contact the first locating block, and the injection device is in an injection end position. That is, the displacement distance of the push rod in the barrel is determined by the added thickness brought about by the combination of the second locating block and the first locating block.

Further, the top of the first locating block extends upward to form a third protrusion, and an end surface of at least one side of the third protrusion is provided with a guide groove. An end surface of the second locating block facing the first locating block is provided with a recess, and an inner wall of the recess is provided with guide protrusions in equal number and corresponding position to the guide grooves. The guide protrusion is slidably connected to the guide groove. With the slidable connection, the injection device is simple in structure and lower in manufacturing cost. Moreover, during microinjection, the doctor can easily separate the second locating block from the first locating block so as to expose the second stopping portion, so that the thumb rest can abut against the second stopping portion.

Further, a bottom of the recess is provided with a through slot for the push rod to run through. The rod body of the push rod runs through the through slot, which facilitates the movement of the push rod.

Further, an outer side end surface of the second locating block is provided with an anti-slip rib. The anti-slip rib is convenient for the doctor and other users to take the locating block, and can avoid slipping.

Further, the third protrusion is a mounting portion of the second locating block. The second locating block includes a cushion portion and an insert portion, and the recess is located between the cushion portion and the insert portion. The second locating block is detachably mounted on the mounting portion of the first locating block through the insert portion.

The disclosure further provides a syringe with a micro assistor (stopping member). The micro assistor includes a first locating block and a second locating block. The first locating block is arranged on a flange of the syringe, and a side of the first locating block close to a thumb rest of the syringe protrudes to form a mounting portion. The second locating block is detachably mounted on the mounting portion, and the second locating block forms a cushion portion between the thumb rest of the syringe and the mounting portion, which has a thickness matched with an injection distance. The second locating block further has an insert portion having the same thickness as the cushion portion, and the second locating block forms a recess between the cushion portion and the insert portion.

It can be understood that the recess divides the second locating block into two portions, namely a first portion and a second portion. When the first portion is inserted into the mounting portion, the first portion is the insert portion, and the second portion is the cushion portion. When the second portion is inserted into the mounting portion, the second portion is the insert portion, and the first portion is the cushion portion. That is, the cushion portion and the insert portion are interchangeable. According to specific conditions, the portion inserted into the mounting portion is the insert portion, and the portion abutting against the thumb rest is the cushion portion. The thickness of the cushion portion is the distance which the push rod can move from the start of injection to the end of injection, which is equivalent to controlling the injection dose through the thickness of the cushion portion.

Further, an end of the mounting portion close to the thumb rest protrudes to form a guide rail fitted with the recess, and a guide slot is arranged below the guide rail. When the second locating block is mounted on the mounting portion of the first locating block, the insert portion is located in the guide slot.

Further, a thickness of the cushion portion and/or the insert portion is not greater than a width of the guide slot. The cushion portion and the insert portion are interchangeable.

The thickness of the insert portion is not greater than the width of the guide slot, so that the insert portion can be smoothly inserted into the guide slot. Therefore, as long as the thickness of the cushion portion and/or the insert portion of the second locating block is less than the width of the guide slot, it can be inserted into the guide slot.

In some embodiments, the thickness of the cushion portion may be greater than the width of the guide slot, and the thickness of the insert portion may be less than the width of the guide slot, so that the second locating block can be inserted into the guide slot through the insert portion. Although the cushion portion cannot be inserted into the guide slot, it can still be used for abutting against the thumb rest of the push rod so as to control the displacement distance of the push rod from the start of injection to the end of injection.

In some embodiments, the thicknesses of both the cushion portion and the insert portion may be respectively less than the width of the guide slot, so that both the insert portion and the cushion portion of the second locating block can be inserted into the guide slot respectively. If the insert portion is inserted into the guide slot, then the cushion portion abuts against the thumb rest. If the cushion portion is inserted into the guide slot, then the insert portion abuts against the thumb rest.

In some embodiments, the thicknesses of both the cushion portion and the insert portion are respectively less than the width of the guide slot, but the two portions have different thicknesses. For example, the thickness of the first portion is a, and the thickness of the second portion is b. When it is required to control the displacement distance of the push rod from the start of injection to the end of injection to be a, then the first portion is selected as the cushion portion, and the second portion is inserted into the guide slot. When it is required to control the displacement distance of the push rod from the start of injection to the end of injection to be b, then the second portion is selected as the cushion portion, and the first portion is inserted into the guide slot.

In some embodiments, the thicknesses of both the cushion portion and the insert portion are respectively less than the width of the guide slot, and the two portions have the same thickness. Then, selection of either portion as the cushion portion or the insert portion has the same effect, and the displacement distance of the push rod controlled is the same.

Further, the cushion portion and the insert portion have the same thickness.

Since the cushion portion and the insert portion have the same thickness and the thickness is the required injection distance, whether the second locating block is mounted in the forward direction or in the reverse direction, the distance of the mounted second locating block protruding out of the mounting portion remains unchanged, which makes the second locating block more convenient to mount. That is, when mounting the second locating block, there is no need to consider whether it is mounted in the forward direction or in the reverse direction, which effectively improves the mounting efficiency of the second locating block and the mounting convenience.

Further, the whole second locating block is U-shaped, and has a lateral insertion slot in an insertion fit with the push rod. The first locating block is provided with a push rod dodging slot in an insertion fit with the push rod, and also provided with a flange insertion slot matched with the flange.

The second locating block and the first locating block can respectively clamp the push rod. When the second locating block is inserted into the guide rail of the first locating block, the direction of the U shape may be either consistent with or opposite to that of the first locating block. As long as the second locating block can be inserted, the displacement distance of the push rod from the start of injection to the end of injection can be fixed and controlled.

In some embodiments, preferably, when the second locating block is inserted into the guide rail of the first locating block, the direction of the U shape is consistent with that of the first locating block so as to improve the mounting stability of the second locating block.

Preferably, an outer side wall of the second locating block is provided with two hand-held members, and the two hand-held members are arranged symmetrically, so that the operator can clamp the two hand-held members with fingers to mount and demount the second locating block.

Preferably, the hand-held member includes a hand-held portion and two connecting arms arranged on the hand-held portion at an interval, and the hand-held portion is connected to the second locating block through the connecting arms. Since there is a notch between the two connecting arms of the hand-held member, the thickness uniformity during molding of the second locating block can be effectively improved.

Preferably, the hand-held portion has a cantilever-shaped press end, and when the two press ends are close to each other, two opposite side walls of the lateral insertion slot open outward, which reduces the difficulty in removing the second locating block.

Preferably, the press end is provided with a press surface, and the press surface is an anti-slip surface, which avoids slipping when being pressed.

Preferably, the whole hand-held portion is wavy, which is more beautiful.

Preferably, a side wall of the second locating block is provided with a thinning slot for facilitating the opening of the lateral insertion slot, which effectively reduces the difficulty in opening the lateral insertion slot.

Preferably, an inner wall of the lateral insertion slot is provided with first clamping members, and an outer wall of the mounting portion is provided with second clamping members clamped with the first clamping members. Preferably, one of each of the first clamping members and each of the second clamping members is a clamping protrusion, and the other is a clamping groove.

In order to achieve one of the above objectives, a third technical solution of the disclosure is as follows:

A method for using an injection device, applied to the above injection device, includes: mounting a stopping member on a push rod or a barrel such that a thumb rest or a flange is aligned with a first stopping portion;
pressing the push rod such that the thumb rest or the flange abuts against the first stopping portion, where at this time, the injection device is in an injection start position;
aligning the thumb rest or the flange with a second stopping portion; and
pressing the push rod such that the thumb rest or the flange abuts against the second stopping portion, where at this time, the injection ends, and a gap is left between a piston and a bottom of the barrel.

There is no need to start and end the injection according to graduation marks. Therefore, there is no need to read the value on the graduation mark, and the graduation marks can be removed. The method for using an injection device is applicable to both an ordinary syringe and a prefilled syringe.

In order to achieve one of the above objectives, a fourth technical solution of the disclosure is as follows:

A method for using an injection device without changing an original structure of a syringe includes:
c: mounting a stopping member on a push rod or a barrel such that a thumb rest or a flange is aligned with a first stopping portion;
d: pressing the push rod such that the thumb rest or the flange abuts against the first stopping portion, where at this time, the injection device is in an injection start position;
e: aligning the thumb rest or the flange with a second stopping portion; and
f: pressing the push rod such that the thumb rest or the flange abuts against the second stopping portion, where at this time, the injection ends, and a gap is left between a piston and a bottom of the barrel.

There is no need to start and end the injection according to graduation marks. Therefore, there is no need to read the value on the graduation mark, and the graduation marks can be removed. The method for using an injection device is applicable to both an ordinary syringe and a prefilled syringe.

Further, the method includes:
a: pressing the push rod until the piston abuts against the bottom of the barrel: evacuating air inside the barrel;
b: putting a part of a needle of the injection device into a medication to be withdrawn, and then pulling the push rod back such that the piston is separated from the bottom of the barrel and the medication is drawn into the barrel, where at this time, the medication is located in a space between the bottom in the barrel and the piston;
c: clamping the push rod with the stopping member, and allowing an end provided with the first stopping portion to be aligned with the flange and the other end to abut against the thumb rest;
d: pressing the push rod such that the flange abuts against the first stopping portion, where at this time, the injection device is in the injection start position;
e: rotating the push rod or the stopping member such that the second stopping portion is aligned with the flange and the first stopping portion is misaligned with the flange; and
f: pressing the push rod such that the flange abuts against the second stopping portion, where at this time, the injection ends, and a gap is left between the piston and the bottom of the barrel.

Further, f: when the flange abuts against the second stopping portion, the first stopping portion is located on two sides of the flange.

When the push rod or the stopping member is rotated such that the second stopping portion is aligned with the flange, the flange is not in the moving path of the first stopping portion, or the flange has been staggered with the first stopping portion. When the push rod is pressed such that the flange abuts against the second stopping portion, the first stopping portion stays at the two narrow sides on the flange, so as not to prevent the flange from abutting against the second stopping portion.

Further, the method includes:
a: pressing the push rod until the piston abuts against the bottom of the barrel: evacuating air inside the barrel;
b: putting a part of a needle of the injection device into a medication to be withdrawn, and then pulling the push rod back such that the piston is separated from the bottom of the barrel and the medication is drawn into the barrel, where at this time, the medication is located in a space between the bottom in the barrel and the piston;
c: slidably connecting a second locating block to a first locating block, and then, allowing the first locating block to be clamped with the flange of the barrel, with the first stopping portion facing the thumb rest;
d: pressing the push rod such that the thumb rest abuts against the first stopping portion, where at this time, the injection device is in the injection start position;
e: removing the second locating block such that the second stopping portion exposes and faces the thumb rest; and
f: pressing the push rod such that the thumb rest abuts against the second stopping portion, where at this time, the injection ends, and a gap is left between the piston and the bottom of the barrel.

The disclosure further provides a method for using the injection device without changing an original structure of a syringe. According to the method, f: when the flange (8) abuts against the second stopping portion (52), the first stopping portion (51) is located on two sides of the flange (8).

Further, the method includes: a: pressing the push rod (2) until the piston (3) abuts against the bottom of the barrel (1); b: putting a part of a needle of the injection device into a medication to be withdrawn, and then pulling the push rod (2) back such that the piston (3) is separated from the bottom of the barrel (1) and the medication is drawn into the barrel (1); c: slidably connecting a second locating block (504) to a first locating block (503), and then, allowing the first locating block (503) to be clamped with the flange (8) of the barrel (1), with the first stopping portion (51) facing the thumb rest (4); d: pressing the push rod (2) such that the thumb rest (4) abuts against the first stopping portion (51), where at this time, the injection device is in the injection start position: e: removing the second locating block (504) such that the second stopping portion (52) exposes and faces the thumb rest (4); and f: pressing the push rod (2) such that the thumb rest (4) abuts against the second stopping portion (52), where at this time, the injection ends, and a gap is left between the piston (3) and the bottom of the barrel (1).

The disclosure has the following beneficial effects:

According to the injection device of the disclosure, the injection start position and the injection end position are no longer based on the graduation marks, and can be set more randomly and freely. As long as the fixed distance a between the first stopping portion and the second stopping portion is determined, the number of tolerances can be greatly reduced, thereby improving the injection accuracy and meeting the requirement of microinjection for high accuracy.

In the meanwhile, since the injection start position and the injection end position are no longer based on the graduation marks, there is no need to read the graduation marks, so that there is no reading error caused by light refraction of the barrel, thereby further improving the injection accuracy.

Moreover, since the injection end position can be set more randomly and freely, when the injection ends, a gap may be left between the piston and the bottom of the barrel, so that the piston will not squeeze the bottom of the barrel and slightly deform. Therefore, the residual medication in a tapper cavity will not be squeezed out for injection, which further improves the microinjection accuracy.

Further, the injection device can realize accurate microinjection without changing the original structure of the syringe, so that an ordinary syringe can also be used for microinjection and there is no need to purchase a special syringe for microinjection, thereby greatly reducing the implementation cost of microinjection.

Furthermore, since there is no need to change the original structure of the syringe, a prefilled syringe can also be combined with the injection device and method to realize microinjection.

Furthermore, the disclosure further provides the syringe with a micro assistor. Since the injection start position and the injection end position are respectively determined by the second locating block and the first locating block, the problem of insufficient injection accuracy caused by error accumulation is effectively avoided. Since the cushion portion and the insert portion have the same thickness and the thickness is the required injection distance, whether the second locating block is mounted in the forward direction or in the reverse direction, the distance of the mounted second locating block protruding out of the mounting portion remains unchanged, which makes the second locating block more convenient to mount.

Since the lateral insertion slot of the second locating block is a through slot and the outer wall of the second locating block is provided with the two hand-held members, when the operator presses the press surfaces of the two hand-held members, the two side walls of the lateral insertion slots will open outward, which effectively reducing the difficulty in removing the second locating block.

Based on the above, the injection device without changing an original structure of a syringe and the method for using the same can achieve many objectives.

### BRIEF DESCRIPTION OF THE DRAWINGS

FIG. 1 is a schematic structural view of a microsyringe with a simple structure in the prior art;

FIG. 2 is a schematic structural view I of the injection device according to Example 1 of the disclosure;

FIG. 3 is a schematic structural view II of the injection device according to Example 1 of the disclosure;

FIG. 4 is a schematic structural view III of the injection device according to Example 1 of the disclosure;

FIG. 5 is a schematic structural view IV of the injection device according to Example 1 of the disclosure;

FIG. 6 is a schematic structural view I of the injection device according to Example 2 of the disclosure;

FIG. 7 is a schematic structural view II of the injection device according to Example 2 of the disclosure;

FIG. 8 is a schematic structural view of a locating block according to Example 2 of the disclosure;

FIG. 9 is a schematic structural view III of the injection device according to Example 2 of the disclosure;

FIG. 10 is a three-dimensional view of a syringe with a micro assistor according to Example 4 of the disclosure;

FIG. 11 is an isometric sectional view of the syringe with a micro assistor according to Example 4 of the disclosure;

FIG. 12 is a three-dimensional view of a first locating block according to Example 4 of the disclosure;

FIG. 13 is a three-dimensional view of a second locating block from a first viewing angle according to Example 4 of the disclosure;

FIG. 14 is a three-dimensional view of the second locating block from a second viewing angle according to Example 4 of the disclosure;

FIG. 15 is a top view of the second locating block according to Example 4 of the disclosure; and

FIG. 16 is a diagram showing the preparation process before using the syringe with a micro assistor according to Example 4 of the disclosure.

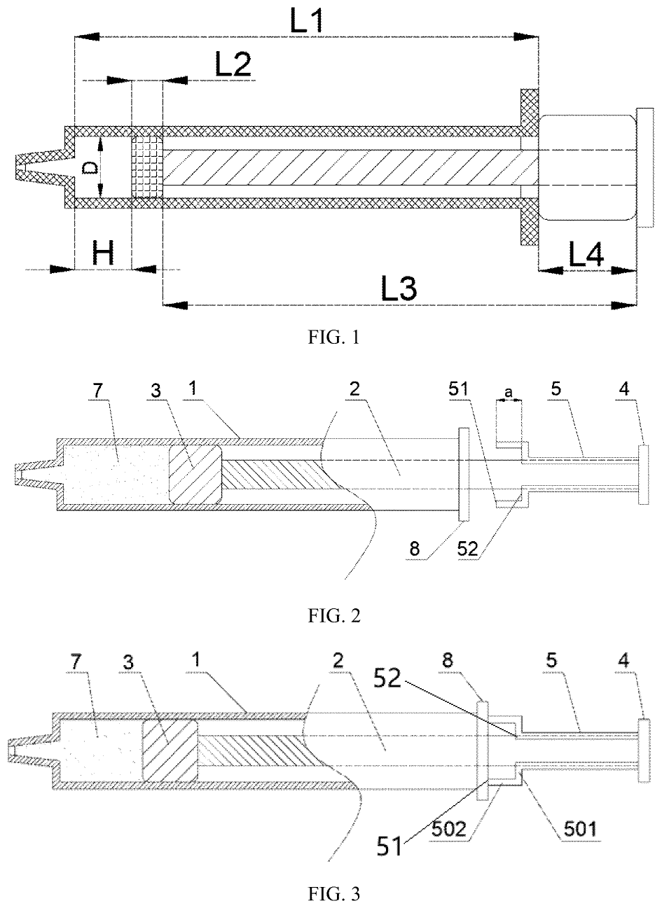

1—barrel, 2—push rod, 3—piston, 4—thumb rest, 5—stopping member, 7—medication, 8—flange, 9—hand-held portion, 11—guide groove, 12—recess, 13—guide protrusion, 14—through slot, 15—anti-slip rib; 51—first stopping portion, 52—second stopping portion, 501—first protrusion, 502—second protrusion, 503—first locating block, 504—second locating block, 505—third protrusion.

### DETAILED DESCRIPTION OF THE INVENTION

### Syringe

The syringe of the disclosure is a conventional injection tool, and the process of withdrawing or injecting a gas or liquid through a needle is called injection. The syringe includes a barrel with a small hole at the front end, and a matched push rod with a piston, and is used for injecting a small amount of liquid into areas inaccessible by other methods or withdrawing it from those areas. When the push rod is pulled out, the liquid or gas is drawn into the barrel through the small hole at the front end. When the push rod is pushed into the barrel, the liquid or gas is squeezed out. The existing syringes that can realize quantitative microinjection are very complicated in structure, and usually need to be controlled by plenty of electrical elements, so the cost is high. The syringe provided by the disclosure can easily realize accurate quantitative microinjection by adding a stopping member without changing the original structure of the syringe. This syringe may be any conventional syringe, which can control the injection dose simply by using a stopping member with a size matched with that of the syringe. Compared with electrical elements in the prior art, the stopping member is more simple in structure, which reduces the use cost and makes the whole microinjection operation more simple and more convenient.

The piston is elastic, when the piston contacts the bottom of the barrel and deforms, the quantification process is prone to errors. Therefore, preferably, a gap is left between the piston and the bottom of the barrel, that is, the injection ends before the piston comes into contact with the bottom of the barrel, thereby improving the control accuracy of quantitative injection. Of course, if the sliding distance of the push rod can be controlled accurately such that the force of the piston coming into contact with the bottom of the barrel every time is consistent, that is, the change in displacement caused by the elasticity of the piston is reproducible in each control process, even if the piston contacts the bottom of the barrel, accurate quantification can still be realized, which, however, will increase the control difficulty and is not a preferred method. Preferably, the injection is ended before the piston comes into contact with the bottom of the barrel, which is more convenient for control and accurate quantification.

Since the barrel of the syringe is provided with the flange and the top end of the push rod is provided with the thumb rest, the displacement distance of the push rod can be controlled by making full use of two opposite surfaces on the flange and the thumb rest. The stopping member is preferably arranged on the push rod between the flange and the thumb rest. While the push rod is pushed, the stopping member stops the displacement of the push rod by making contact with the flange or the thumb rest, thereby controlling the injection dose. The flange here does not mean that the top end of the barrel has a curled structure, but that it has a surface connected with the barrel and folded outward. This surface may be flat or curved. If this surface is curved, the stopping member may be provided with a matched curved surface to be stopped by or combined with this surface. If this surface is flat, the stopping member may also be stopped by or combined with this surface through a flat surface or a non-flat surface. Similarly, the surface of the thumb rest opposite to the flange may also be flat or curved. As long as the displacement of the push rod can be limited by making the thumb rest or the flange stopped by the stopping member, the injection dose can be controlled.

### Stopping Member

The stopping block may be fixed on the push rod, on the barrel, or between the push rod and the barrel. As long as the stopping block can be fixed and can play a certain stopping role on the push rod when the push rod is pushed (for example, the stopping member is elastic, and the push rod is only allowed to move within its elasticity allowable range and not allowed to exceed this range), it can control the displacement distance of the push rod in the barrel, thereby controlling the injection dose. In order to control the movement of the push rod more accurately, the stopping member is fixedly fixed on the push rod or on the barrel, which can avoid affecting the movement of the push rod relative to the barrel. When the stopping block is located on the push rod, it may be located either on the push rod outside the barrel or on the push rod inside the barrel. As long as the stopping block can limit the sliding distance of the push rod in the barrel, the injection dose can be accurately controlled. In order to facilitate use, more preferably, the stopping member is fixed on the push rod outside the barrel, so that the stopping member can be directly arranged on the original syringe, which is more convenient and controllable.

By improving the structure of the stopping member, the stopping member is provided with a first stopping portion and a second stopping portion. A distance between the first stopping portion and the second stopping portion is a fixed distance a. The push rod is moved, and when the push rod is stopped by the first stopping portion, the injection device is in an injection start position; and when the push rod is stopped by the second stopping portion, the injection ends. Moreover, with the flange of the barrel and the thumb rest of the push rod of the syringe, the stopping block stops the push rod respectively through the two opposite surfaces on the flange and the thumb rest, which is more direct and more controllable. The first stopping portion and the second stopping portion may not be flat, as long as several points protruding from the first stopping portion or the second stopping portion can abut against the thumb rest or the flange to make the push rod stop moving. Of course, the first stopping portion and the second stopping portion are preferably structures matched with the thumb rest or the flange. When the surface of the thumb rest or the flange for abutting is flat, the first stopping portion and the second stopping portion are also preferably flat.

The stopping member may be an integrated structure, and different surfaces of the integrated structure are used for stopping the push rod: or the stopping member may be split into a plurality of portions, and the displacement of the push rod is controlled by removing different split portions.

When the stopping member is an integrated structure, the stopping member clamps the push rod as a whole, and one end of the stopping member abuts against the thumb rest of the push rod; and the other end extends to form a protrusion, and different positions of the protrusion respectively form a first stopping portion and a second stopping portion which respectively abut against the flange, so that the injection distance of the push rod is controlled. For example, the other end of the stopping member extends outward along a radial direction of the push rod to form a first protrusion, and an end of the first protrusion away from the stopping member extends toward the barrel to form a second protrusion. An end surface of the second protrusion facing the barrel is the first stopping portion. An end surface of the first protrusion facing the barrel is the second stopping portion. When the flange abuts against the first stopping portion, the injection device is in the injection start position. When the flange abuts against the second stopping portion, the injection ends. In addition, one end of the stopping member abuts against the flange, the other end extends to form a protrusion, and different positions of the protrusion respectively form a first stopping portion and a second stopping portion which respectively abut against the flange, so that the injection distance of the push rod is controlled, which can also control the displacement of the push rod.

When the stopping member includes a plurality of portions, it is more convenient to control the displacement of the push rod as compared with the integrated structure. This is because the stopping member in the integrated structure can only stop the thumb rest or the flange through different positions of the structure. The integrated structure is more complicated in design and difficult to control, and once a protrusion is not clamped successfully, the control of the injection dose will fail. When the stopping member is designed as a split structure, it is only required to remove a portion of the structure, and the displacement distance of the push rod is limited through the stopping effect before and after the removal, which is easier to operate and more accurate in the control of the displacement distance and can realize microinjection with higher accuracy.

When the stopping member includes two portions, it includes a first locating block and a second locating block, and the first locating block and the second locating block are connected and separated detachably. For example, the first locating block is clamped on the flange, and a top of the first locating block is connected with the second locating block; an end surface of the second locating block facing the thumb rest is the first stopping portion, and an end surface of a top of the first locating block facing the thumb rest is the second stopping portion; when the thumb rest abuts against the first stopping portion, the injection device is in the injection start position; and when the thumb rest abuts against the second stopping portion, the injection ends. For another example, the first locating block is clamped on the thumb rest, and a top of the first locating block is connected with the second locating block; an end surface of the second locating block facing the flange is the first stopping portion, and an end surface of a top of the first locating block facing the flange is the second stopping portion; when the flange abuts against the first stopping portion, the injection device is in the injection start position; and when the flange abuts against the second stopping portion, the injection ends. That is, when the first locating block and the second locating block are combined, the injection device is in the injection start position. After the second locating block is disassembled and taken out, the push rod is pushed to contact the first locating block, and the injection device is in the injection end position. The displacement distance of the push rod in the barrel is determined by the added thickness brought about by the combination of the second locating block and the first locating block, that is, the thickness of the combination of the second locating block and the first locating block minus the thickness of the first locating block alone is the displacement distance of the push rod in the barrel.

It can be understood that the stopping member may also include a plurality of portions, for example, three portions. In this case, the displacement of the push rod can be controlled twice. For example, if 10 μl and 20 μl of medication need to be injected respectively, the injection of 10 μl can be realized through the removal of one portion, and the injection of 20 μl can be realized through the removal of another portion. If 30 μl of medication needs to be injected at one time, then these two portions can be removed at the same time. By analogy, the stopping member may include different numbers of portions according to needs as long as the stopping member can be arranged between the flange and the thumb rest and has a satisfactory size, and the injection dose can be controlled by determining which portions need to be removed according to needs.

When the stopping member includes two portions, the structures of the first locating block and the second locating block can be improved to make their combination more stable and their removal more convenient. For example, a guide rail and a recess may be respectively arranged between the first locating block and the second locating block such that the first locating block and the second locating block are combined into an integrated structure. According to the disclosure, the top of the first locating block extends upward to form a third protrusion, and an end surface of at least one side of the third protrusion is provided with a guide groove; an end surface of the second locating block facing the first locating block is provided with a recess, and an inner wall of the recess is provided with guide protrusions in equal number and corresponding position to the guide grooves; and the guide protrusion is slidably connected to the guide groove. With the slidable connection, the injection device is simple in structure and lower in manufacturing cost. Moreover, during microinjection, the doctor can easily separate the second locating block from the first locating block so as to expose the second stopping portion, so that the thumb rest can abut against the second stopping portion. Of course, the third protrusion may also be provided on the second locating block so as to facilitate the combination with and separation from the first locating block, which can also be realized. However, providing the third protrusion on the first locating block at a lower position is more in line with the operating habits and less prone to errors.

Since both the first locating block and the second locating block need to be clamped with the push rod, a bottom of the recess is provided with a through slot for the push rod to run through. The rod body of the push rod runs through the through slot, which facilitates the movement of the push rod. Moreover, an outer side end surface of the second locating block that needs to be removed may be provided with an anti-slip rib. The anti-slip rib is convenient for the doctor and other users to take the locating block, and can avoid slipping.

The disclosure further provides another stopping member, which also includes two portions, namely a first locating block and a second locating block: the first locating block is arranged on a flange of the syringe, and a side of the first locating block close to a thumb rest of the syringe protrudes to form a mounting portion (which is equivalent to the third protrusion); and the second locating block is detachably mounted on the mounting portion. The second locating block is divided into an upper portion and a lower portion, namely a cushion portion and an insert portion, and the recess is located between the cushion portion and the insert portion. The second locating block is detachably mounted on the mounting portion of the first locating block through the insert portion. However, the cushion portion and the insert portion here are not fixed, but interchangeable. That is, the recess in the middle divides the second locating block into two portions, namely a first portion and a second portion. When the first portion is inserted into the mounting portion, the first portion is the insert portion, and the second portion is the cushion portion. When the second portion is inserted into the mounting portion, the second portion is the insert portion, and the first portion is the cushion portion. That is, the cushion portion and the insert portion are interchangeable. According to specific conditions, the portion inserted into the mounting portion is the insert portion, and the portion abutting against the thumb rest is the cushion portion. The thickness of the cushion portion is the distance which the push rod can move from the start of injection to the end of injection, that is, the thickness of the cushion portion is the added thickness brought about by the combination of the second locating block and the first locating block, which is equivalent to controlling the displacement distance of the push rod in the barrel through the thickness of the cushion portion so as to control the injection dose.

The mounting portion of the first locating block is provided with a guide groove, and the guide groove includes a guide rail which is formed by protruding from an end close to the thumb rest and fitted with the recess, and a guide slot below the guide rail. When the second locating block is mounted on the mounting portion of the first locating block, the insert portion is located in the guide slot. Therefore, the thickness of the insert portion is not greater than the width of the guide slot, so that the insert portion can be smoothly inserted into the guide slot. Therefore, as long as the thickness of the cushion portion and/or the insert portion of the second locating block is less than the width of the guide slot, it can be inserted into the guide slot. There are many cases for the thicknesses of the cushion portion and the insert portion. For example, the thickness of the cushion portion may be greater than the width of the guide slot, and the thickness of the insert portion may be less than the width of the guide slot, so that the second locating block can be inserted into the guide slot through the insert portion. Although the cushion portion cannot be inserted into the guide slot, it can still be used for abutting against the thumb rest of the push rod so as to control the displacement distance of the push rod from the start of injection to the end of injection. Of course, the thickness of the cushion portion should not be too large, which will make the cushion portion unable to be inserted between the flange and the thumb rest, thus making the stopping member unavailable. For example, the thicknesses of both the cushion portion and the insert portion may be respectively less than the width of the guide slot, so that both the insert portion and the cushion portion of the second locating block can be inserted into the guide slot respectively. If the insert portion is inserted into the guide slot, then the cushion portion abuts against the thumb rest. If the cushion portion is inserted into the guide slot, then the insert portion abuts against the thumb rest. In this case, there are also two cases. First, the thicknesses of both the cushion portion and the insert portion are respectively less than the width of the guide slot, but the two portions have different thicknesses. For example, the thickness of the first portion is a, and the thickness of the second portion is b. When it is required to control the displacement distance of the push rod from the start of injection to the end of injection to be a, then the first portion is selected as the cushion portion, and the second portion is inserted into the guide slot. When it is required to control the displacement distance of the push rod from the start of injection to the end of injection to be b, then the second portion is selected as the cushion portion, and the first portion is inserted into the guide slot. Second, the thicknesses of both the cushion portion and the insert portion are respectively less than the width of the guide slot, and the two portions have the same thickness. The selection of either the first portion or the second portion as the cushion portion/insert portion has the same effect, and the displacement distance of the push rod controlled is the same. When the cushion portion and the insert portion have the same thickness and the thickness is the required injection distance, whether the second locating block is mounted in the forward direction or in the reverse direction, the distance of the mounted second locating block protruding out of the mounting portion remains unchanged, which makes the second locating block more convenient to mount. That is, when mounting the second locating block, there is no need to consider whether it is mounted in the forward direction or in the reverse direction, which effectively improves the mounting efficiency of the second locating block and the mounting convenience.

When the first locating block and the second locating block are combined, they are located between the flange and the thumb rest and clamp the push rod. The whole second locating block is U-shaped, and has a lateral insertion slot in an insertion fit with the push rod. The first locating block is provided with a push rod dodging slot in an insertion fit with the push rod, and also provided with a flange insertion slot matched with the flange. The second locating block and the first locating block can respectively clamp the push rod. When the second locating block is inserted into the guide rail of the first locating block, the direction of the U shape may be either consistent with or opposite to that of the first locating block. As long as the second locating block can be inserted, the displacement distance of the push rod from the start of injection to the end of injection can be fixed and controlled. Preferably, when the second locating block is inserted into the guide rail of the first locating block, the direction of the U shape is consistent with that of the first locating block so as to improve the mounting stability of the second locating block. Moreover, this is more in line with the operating habits and less prone to errors.

### Specific Embodiments

The disclosure will be further described in detail below with reference to the accompanying drawings and examples. It should be noted that the following examples are intended to facilitate the understanding of the disclosure and do not limit the disclosure in any way.

In order to make the objectives, technical solutions and advantages of the disclosure clearer, the disclosure will be further described in detail below with reference to the accompanying drawings and examples. It should be understood that the specific examples described herein are merely illustrative of the disclosure and are not intended to limit the disclosure.

On the contrary, the disclosure covers any substitutions, modifications, equivalent methods and solutions within the spirit and scope of the disclosure defined by the claims. Further, in order to make the public have a better understanding of the disclosure, some specific detailed parts are described in detail in the following detailed description of the disclosure. Those skilled in the art can fully understand the disclosure even without the description of these detailed parts.

It should be noted that when two elements are “slidably connected”, the two elements may be directly connected or there may be an intervening element. On the contrary, when an element is defined to be “directly” “on” another element, there is no intervening element. The terms “on”, “under” and similar expressions used herein are for illustrative purposes only.

Unless otherwise defined, all technical and scientific terms used herein have the same meaning as commonly understood by those skilled in the art of the disclosure. The terms used herein are only for the purpose of describing specific examples and are not intended to limit the disclosure.

### Example 1: Injection Device without Changing Original Structure of Syringe and Method for Using Same

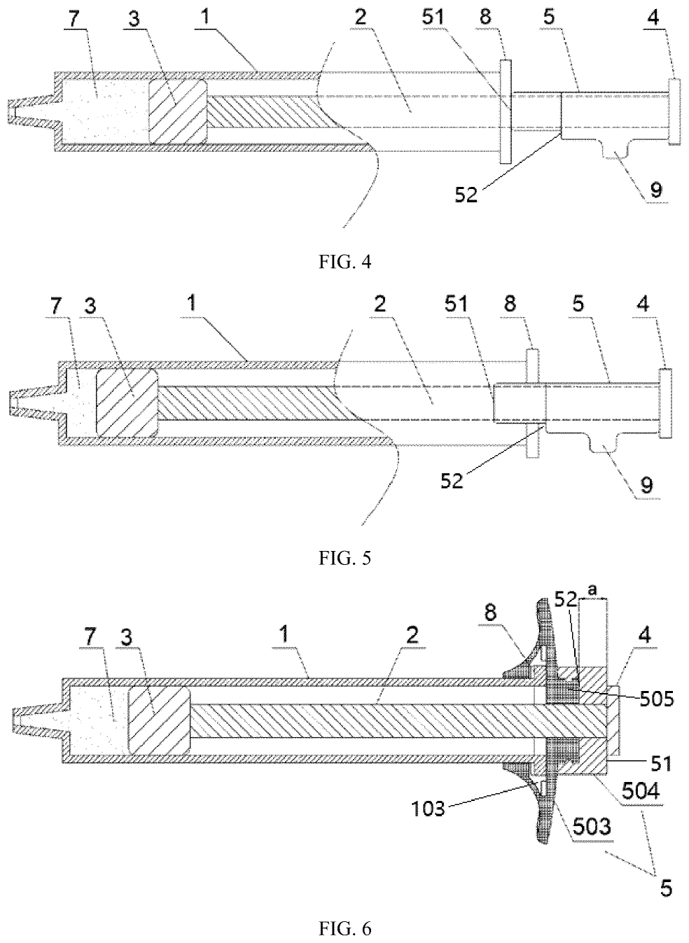

Referring to FIG. 2 to FIG. 5, an injection device without changing an original structure of a syringe includes a barrel 1 with a flange 8 and a push rod 2 slidably connected in the barrel 1. One end of the push rod 2 is provided with a piston 3, and the other end is provided with a thumb rest 4. The piston 3 is located inside the barrel 1, and the thumb rest 4 is located outside the barrel 1.

The push rod 2 is detachably clamped by the stopping member 5, the stopping member 5 is a stopping clamp, and one end of the stopping member 5 abuts against the thumb rest 4. The other end of the stopping member 5 extends outward along a radial direction of the push rod 2 to form a first protrusion 501, and an end of the first protrusion 501 away from the stopping member 5 extends toward the barrel 1 to form a second protrusion 502. An end surface of the second protrusion 502 facing the barrel 1 is a first stopping portion 51. An end surface of the first protrusion 501 facing the barrel 1 is a second stopping portion 52. The first stopping portion 51 and the second stopping portion 52 are distributed along an axis direction of the push rod 2, and a distance between the first stopping portion 51 and the second stopping portion 52 along this direction is a fixed distance a.

The push rod 2 is moved.

When the flange 8 abuts against the first stopping portion 51, the injection device is in an injection start position; and
when the flange 8 abuts against the second stopping portion 52, the injection ends, and a gap is left between the piston 3 and a bottom of the barrel 1.

An outer wall of the stopping member 5 is provided with a hand-held portion 9.

A method for using an injection device without changing an original structure of a syringe includes:
a: pressing the push rod 2 until the piston 3 abuts against the bottom of the barrel 1; evacuating air inside the barrel 1;
b: putting a part of a needle of the injection device into a medication to be withdrawn, and then pulling the push rod 2 back such that the piston 3 is separated from the bottom of the barrel 1 and the medication is drawn into the barrel 1, where at this time, the medication 7 is located in a space between the bottom in the barrel 1 and the piston 3;
c: clamping the push rod 2 with the stopping member 5, and allowing an end provided with the first stopping portion 51 to be aligned with the flange 8 and the other end to abut against the thumb rest 4;
d: pressing the push rod 2 such that the flange 8 abuts against the first stopping portion 51, where at this time, the injection device is in the injection start position, and at this time, a small amount of medication is discharged, which indicates that the air in the barrel 1 has been evacuated and injection can be started;
e: rotating the push rod 2 or the stopping member 5 such that the second stopping portion 52 is aligned with the flange 8 and the first stopping portion 51 is misaligned with the flange 8, where when rotating the stopping member 5, the stopping member 5 may be slightly loosened, rotated and then allowed to clamp the push rod again; and
f: pressing the push rod 2 such that the flange 8 abuts against the second stopping portion 52, where at this time, the injection ends, and a gap is left between the piston 3 and the bottom of the barrel 1; and when the flange 8 abuts against the second stopping portion 52, the first stopping portion 51 is located on two sides of the flange 8.

The overall working principle is as follows:

An inner diameter of a 0.5 mL barrel specified in the international standard ISO11040-6 (i.e., the inner diameter D of the barrel 1 is 4.7 mm±0.1 mm) is adopted, and this barrel 1 is used to inject 50 microliters±5 microliters (i.e., with an error of 10%) of medication.

The dose V to be injected is known to be 50 microliters (μL). According to the inner diameter D=4.7 mm of the barrel 1, the cross-sectional area S=17.35 mm² of the barrel 1 can be calculated. The distance from the first stopping portion 51 to the second stopping portion 52 is the fixed distance a. The fixed distance a represents the movement distance of the push rod 2, i.e., the height of the injected medication, which is the same as the height H of the medication in the prior art. Then, according to V=S*a, a=2.9 mm can be calculated. The tolerance is ±0.1 mm.

Therefore, at the maximum tolerance:
D=4.7+0.1=4.8 mm, S=π(D/2) 2=18.10 mm², a=2.9+0.1=3.0 mm, V=S*a=54.3 μL;
at the minimum tolerance:
D=4.7−0.1=4.6 mm, S=π(D/2) 2=16.62 mm², a=2.9−0.1=2.8 mm, V=S*a=46.5 μL.

The following table can be obtained:

| | Inner | | Cross- | | | | |
|---|---|---|---|---|---|---|---|
| | diameter | | sectional | Fixed | | | |
| | D of | Tolerance | area | distance | Tolerance | Dose | Injection |
| | syringe/mm | of D/mm | S/mm² | a/mm | of a/mm | V/μL | accuracy |
| Maximum | 4.8 | — | 18.10 | 3.0 | — | 54.3 | 7.9% |
| tolerance | | | | | | | |
| Intermediate | 4.7 | ±0.1 | 17.35 | 2.9 | ±0.1 | 50.3 | — |
| size | | | | | | | |
| Minimum | 4.6 | — | 16.62 | 2.8 | — | 46.5 | −7.6% |
| tolerance | | | | | | | |

As can be seen from the above table, when the injection device is at the maximum tolerance, the injection dose is 54.3 microliters, and when the injection device is at the minimum tolerance, the injection dose is 46.5 microliters. Both values do not exceed the error of 5 microliters. Therefore, the injection device meets the requirement of an error within ±10% when used for microinjection of 50 microliters. Moreover, in the whole process, there is no need to read any graduation marks. The piston 3 does not make contact with the bottom of the barrel 1, so that the piston will not be squeezed to produce slight deformation. Moreover, there is no need to change the original structure of the syringe.

### Example 2

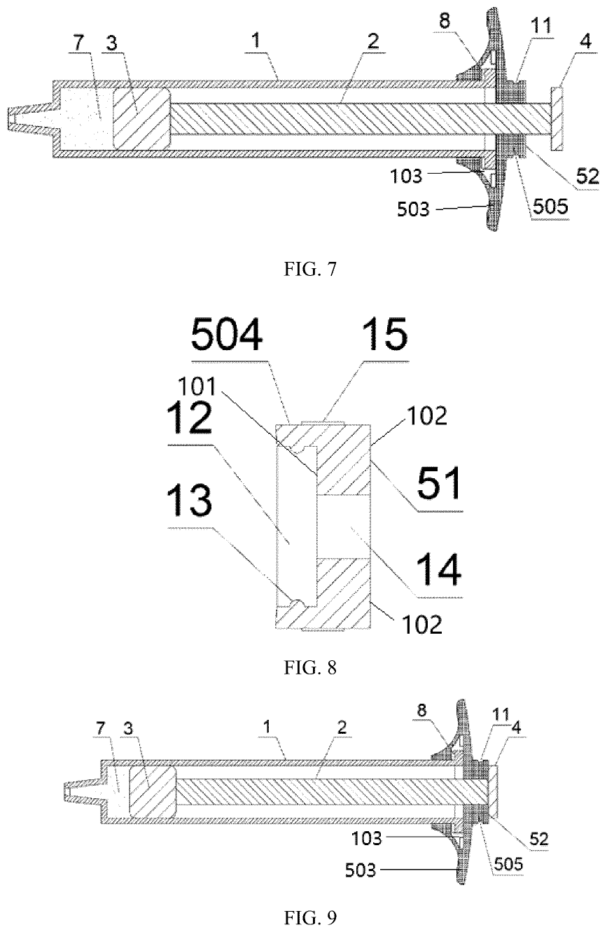

Referring to FIG. 6 to FIG. 9, an injection device without changing an original structure of a syringe includes a barrel 1 with a flange 8 and a push rod 2 slidably connected in the barrel 1. One end of the push rod 2 is provided with a piston 3, and the other end is provided with a thumb rest 4. The piston 3 is located inside the barrel 1, and the thumb rest 4 is located outside the barrel 1. The barrel 1 is detachably connected with a stopping member 5, and the stopping member 5 includes a first locating block 503 and a second locating block 504. The first locating block 503 is provided with a flange clamping slot 103, so that the flange 8 is detachably clamped with the first locating block 503, and a top of the first locating block 503 is detachably connected with the second locating block 504. An end surface of the second locating block 504 facing the thumb rest 4 is a first stopping portion 51, and an end surface of a top of the first locating block 503 facing the thumb rest 4 is a second stopping portion 52. A distance between the first stopping portion 51 and the second stopping portion 52 is a fixed distance a.

The push rod 2 is moved.

When the thumb rest 4 abuts against the first stopping portion 51, the injection device is in an injection start position; and
when the thumb rest 4 abuts against the second stopping portion 52, the injection ends, and a gap is left between the piston 3 and a bottom of the barrel 1.

The top of the first locating block 503 extends upward to form a third protrusion 505, and an end surface of at least one side of the third protrusion 505 is provided with a guide groove 11. An end surface of the second locating block 504 facing the first locating block 503 is provided with a recess 12, and an inner wall of the recess 12 is provided with guide protrusions 13 in equal number and corresponding position to the guide grooves 11. The guide protrusion 13 is slidably connected to the guide groove 11.

A distance from a bottom end surface 101 in the recess 12 to an end surface 102 of the second locating block 504 facing the thumb rest 4 is also a.

A bottom of the recess 12 is provided with a through slot 14 for the push rod 2 to run through.

An outer side end surface of the second locating block 504 is provided with an anti-slip rib 15.

A method for using an injection device without changing an original structure of a syringe includes:
a: pressing the push rod 2 until the piston 3 abuts against the bottom of the barrel 1; evacuating air inside the barrel 1;
b: putting a part of a needle of the injection device into a medication to be withdrawn, and then pulling the push rod 2 back such that the piston 3 is separated from the bottom of the barrel 1 and the medication is drawn into the barrel 1, where at this time, the medication 7 is located in a space between the bottom in the barrel 1 and the piston 3;
c: slidably connecting the second locating block 504 to the first locating block 503, and then, allowing the first locating block 503 to be clamped with the flange 8 of the barrel 1, with the first stopping portion 51 facing the thumb rest 4;
d: pressing the push rod 2 such that the thumb rest 4 abuts against the first stopping portion 51, where at this time, the injection device is in the injection start position, and at this time, a small amount of medication is discharged, which indicates that the air in the barrel 1 has been evacuated and injection can be started;
e: removing the second locating block 504 such that the second stopping portion 52 exposes and faces the thumb rest 4; aligning the thumb rest 4 with the second stopping portion 52; and
f: pressing the push rod 2 such that the thumb rest 4 abuts against the second stopping portion 52, where at this time, the injection ends, and a gap is left between the piston 3 and the bottom of the barrel 1.

The overall working principle is as follows:

An inner diameter of a 0.5 mL barrel specified in the international standard ISO11040-6 (i.e., the inner diameter D of the barrel 1 is 4.7 mm±0.1 mm) is adopted, and this barrel is used to inject 100 microliters'10 microliters (i.e., with an error of 10%) of medication.

The dose V to be injected is known to be 100 microliters (μL). According to the inner diameter D=4.7 mm of the barrel 1, the cross-sectional area S=17.35 mm² of the barrel 1 can be calculated. The distance from the first stopping portion 51 to the second stopping portion 52 is the fixed distance a. The fixed distance a represents the movement distance of the push rod 2, i.e., the height of the injected medication, which is the same as the height H of the medication in the prior art. Then, according to V=S*a, a=5.8 mm can be calculated. The tolerance is ±0.1 mm.

Therefore, at the maximum tolerance:
D=4.7+0.1=4.8 mm, S=π(D/2) 2=18.10 mm², a=5.8+0.1=5.9 mm, V=S*a=106.8 μL;
at the minimum tolerance;
D=4.7−0.1=4.6 mm, S=π(D/2) 2=16.62 mm², a=5.8−0.1=5.7 mm, V=S*a=94.7 μL.

The following table can be obtained:

| | Inner | | Cross- | | | | |
|---|---|---|---|---|---|---|---|
| | diameter | | sectional | Fixed | | | |
| | D of | Tolerance | area | distance | Tolerance | Dose | Injection |
| | syringe/mm | of D/mm | S/mm² | a/mm | of a/mm | V/μL | accuracy |
| Maximum | 4.8 | — | 18.10 | 3.0 | — | 54.3 | 7.9% |
| tolerance | | | | | | | |
| Intermediate | 4.7 | ±0.1 | 17.35 | 2.9 | ±0.1 | 50.3 | — |
| size | | | | | | | |
| Minimum | 4.6 | — | 16.62 | 2.8 | — | 46.5 | −7.6% |
| tolerance | | | | | | | |

As can be seen from the above table, when the injection device is at the maximum tolerance, the injection dose is 106.8 microliters, and when the injection device is at the minimum tolerance, the injection dose is 94.7 microliters. Both values do not exceed the error of 10 microliters. Therefore, the injection device meets the requirement of an error within ±10% when used for microinjection of 100 microliters. Moreover, in the whole process, there is no need to read any graduation marks. The piston 3 does not make contact with the bottom of the barrel 1, so that the piston will not be squeezed to produce slight deformation. Moreover, there is no need to change the original structure of the syringe.

Moreover, when the syringe is used for injection of a dose V of 50 microliters (μL), the error also does not exceed 5 microliters, even with a higher accuracy than the method provided by Example 1. This is because in Example 1, the alignment of the first stopping portion 51 or the second stopping portion 52 with the flange 8 needs to be observed with naked eyes, and especially in the method shown in FIG. 3, even the alignment of the second stopping portion 52 with the flange 8 needs to be observed with naked eyes, which will inevitably lead to errors in the control of the distance a. Moreover, different operators may produce different results. However, this example adopts the combination of the first locating block 503 and the second locating block 504. The distance a from the first stopping portion 51 to the second stopping portion 52 is actually the distance from the bottom end surface 101 on the second locating block 504 to the end surface 102 facing the thumb rest 4. Both the first locating block and the second locating block will directly abut against the thumb rest 4, so there is no need for observation with naked eyes. Therefore, the distance a will inevitably be controlled more accurately, and the operation is more convenient.

### Example 3

This example is different from Example 1 and Example 2 in that a prefilled syringe is used as the original syringe, and the method for using this syringe includes: c: mounting a stopping member 5 on a push rod 2 or a barrel 1 such that a thumb rest 4 or a flange 8 is aligned with a first stopping portion 51;
d: pressing the push rod 2 such that the thumb rest 4 or the flange 8 abuts against the first stopping portion 51, where at this time, the injection device is in an injection start position, and at this time, a small amount of medication is discharged, which indicates that the air in the barrel 1 has been evacuated and injection can be started;
e: aligning the thumb rest 4 or the flange 8 with a second stopping portion 52, where the thumb rest 4 or the flange 8 can be aligned with the second stopping portion 52 by rotating the stopping member 5 or removing a second locating block 504 in the stopping member 5; and
f: pressing the push rod 2 such that the thumb rest 4 or the flange 8 abuts against the second stopping portion 52, where at this time, the injection ends, and a gap is left between a piston 3 and a bottom of the barrel 1.

The overall structure and the overall working principle in this example are the same as those in Example 1 or Example 2, and thus will not be repeated here.

### Example 4

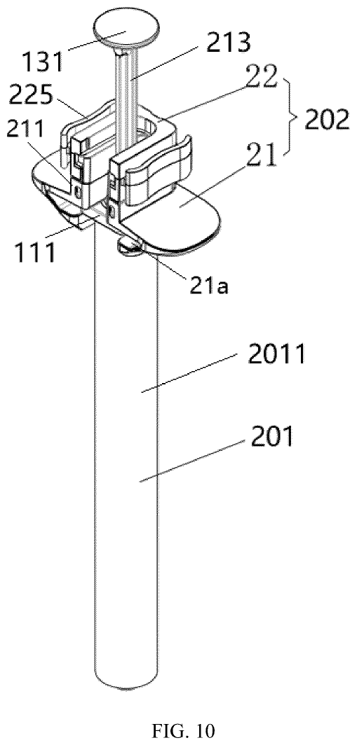

This example relates to a syringe with a micro assistor, as shown in FIG. 10 to FIG. 16, which can accurately control the microinjection of a syringe 201 through a micro assistor 202. For convenience of description, in the following examples, the axis direction of the syringe 201 in the vertical state is defined as the up-down direction.

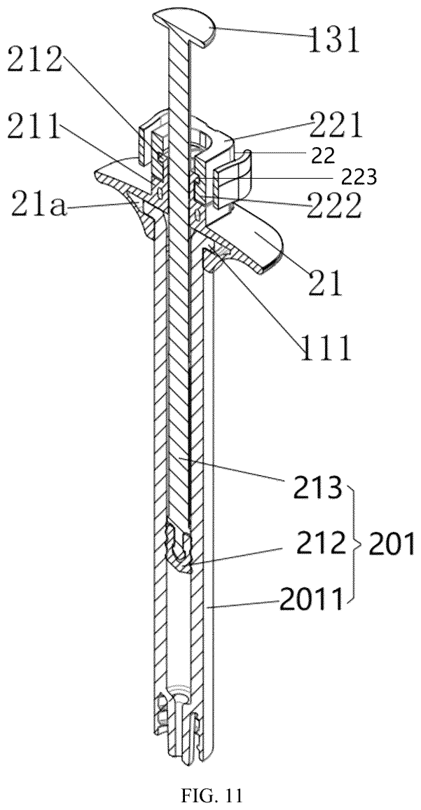

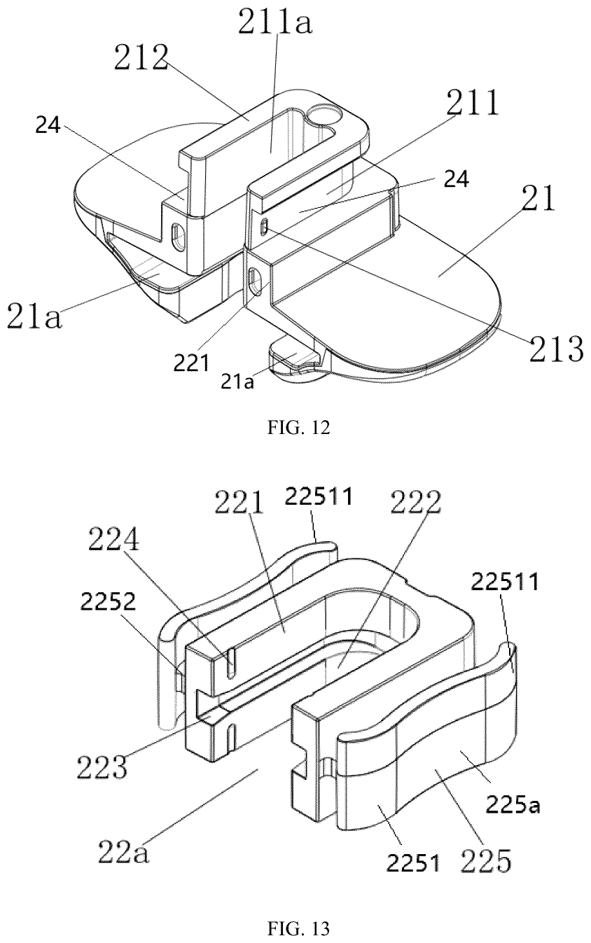

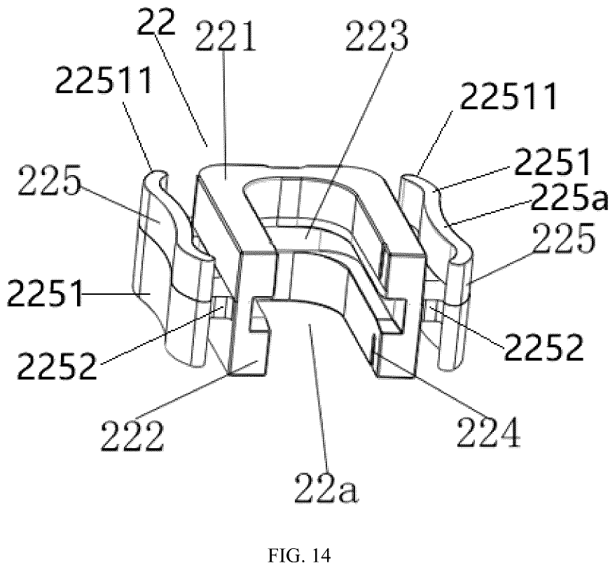

In this application, the syringe 201 is prior art, and its structure is shown in FIG. 11. The syringe 201 includes an injection barrel 2011, a piston 212 and a push rod 213. An upper end of the injection barrel 2011 is an open end, and a plate-like flange 111 is formed at the open end. A lower end of the injection barrel 2011 is a liquid medication inlet/outlet end, which is used for mounting a needle. The piston 212 axially slides in the injection barrel 2011 in a hermetical manner. A lower end of the push rod 213 extends into the injection barrel 2011 and is fixedly connected to the piston 212, and a thumb rest 131 of the syringe is formed at an upper end of the push rod 213 for the operator to press. In this way, the liquid medication can be drawn in or discharged out of the injection barrel 2011 simply by pushing or pulling the push rod 213.

As shown in FIG. 10, FIG. 11, FIG. 12, FIG. 13 and FIG. 14, the micro assistor includes a first locating block 21 and a second locating block 22. The first locating block 21 is arranged on the flange 111 of the syringe 201, and a top end of the first locating block 21 protrudes upward (i.e., protrudes toward a side close to the thumb rest 131 of the syringe) to form a mounting portion 211. The second locating block 22 is detachably mounted on the mounting portion 211, and the second locating block 22 includes a cushion portion 221 and an insert portion 222 at an interval in the up-down direction. The second locating block 22 forms a recess 223 between the cushion portion 221 and the insert portion 222, and a top end (i.e., an end close to the thumb rest 131 of the syringe) of the mounting portion 211 protrudes outward to form a guide rail 212 fitted with the recess 223. The second locating block 22 is fitted and connected with the guide rail 212 on the mounting portion 211 through the recess 223. When the second locating block 22 is mounted on the mounting portion 211 of the first locating block 21, the insert portion 222 is located in the guide slot 24 of the mounting portion 211. The thickness of the insert portion 222 is not greater than the width of the guide slot 24, so that the insert portion 222 can be smoothly inserted into the guide slot 24. Therefore, as long as the thickness of the cushion portion 221 and/or the insert portion 222 of the second locating block 22 is less than the width of the guide slot 24, it can be inserted into the guide slot 24. If the thickness of the cushion portion 221 is greater than the width of the guide slot 24 and the thickness of the insert portion 222 is less than the width of the guide slot 24, then the second locating block can be inserted into the guide slot 24 through the insert portion 222. Although the cushion portion 221 cannot be inserted into the guide slot 24, it can still be used for abutting against the thumb rest 131 of the push rod 213 so as to control the displacement distance of the push rod 213 from the start of injection to the end of injection. The thicknesses of both the cushion portion 221 and the insert portion 222 may be respectively less than the width of the guide slot 24, so that both the insert portion 222 and the cushion portion 221 of the second locating block 22 can be inserted into the guide slot 24 respectively. If the insert portion 222 is inserted into the guide slot 24, then the cushion portion 221 abuts against the thumb rest 131. If the cushion portion 221 is inserted into the guide slot 24, then the insert portion 222 abuts against the thumb rest 131. If the thicknesses of both the cushion portion 221 and the insert portion 222 are respectively less than the width of the guide slot 24 but the two portions have different thicknesses, then the cushion portion 222 and the insert portion 222 may be selected based on their thicknesses according to needs, so that different distances of the push rod 213 from the start of injection to the end of injection can be realized, that is, different injection doses can be realized.

Preferably, the thicknesses of both the cushion portion 221 and the insert portion 222 are respectively less than the width of the guide slot 24, and the two portions have the same thickness. Then, selection of either portion as the cushion portion 221 or the insert portion 222 has the same effect, and the displacement distance of the push rod 213 controlled is the same.

Preferably, the cushion portion 221 and the insert portion 222 have the same thickness, and this thickness is matched with the injection distance a and matched with the width of the guide slot 24. In this case, whether the second locating block 22 is mounted on the mounting portion 211 in the forward direction (i.e., the cushion portion 221 is in the upper position) or in the reverse direction (i.e., the cushion portion 221 is in the upper position), the height of the second locating block 22 protruding out of the top of the mounting portion 211 remains unchanged, so that the operator does not need to consider whether the second locating block 22 is mounted in the forward direction or in the reverse direction, which improves the mounting convenience of the second locating block 22. The first locating block 21 and the flange 111 may be connected either detachably or undetachably, which is not limited. In this example, they are preferably connected detachably, so that the first locating block 21 can be reused.

In a preferred example, as shown in FIG. 11 and FIG. 12, a side wall of the first locating block 21 is provided with a flange insertion slot 21a in an insertion fit with the flange 111, and the flange insertion slot 21a may be elastic such that it can be better fixed on the flange 111, and a side wall of the mounting portion 211 is provided with a push rod dodging slot 211a in communication with the flange insertion slot 21a. The push rod dodging slot 211a has a through slot structure running through from top to bottom, and the direction of the opening of the push rod dodging slot 211a is the same as the direction of the opening of the flange insertion slot 21a. In this way, when the flange 111 of the injection barrel 2011 is laterally inserted into the flange insertion slot 21a, the push rod 213 can smoothly enter the push rod dodging slot 211a and move up and down in the push rod dodging slot 211a, thereby completing the detachable connection between the first locating block 21 and the syringe 1. In this example, the first locating block 21 is a gripping member formed with a handle. In order to ensure the forming quality of the first locating block 21, two ends of the flange insertion slot 21a in the direction perpendicular to the insertion and removal direction need to extend accordingly to improve the thickness uniformity of the first locating block 21.

In a preferred example, as shown in FIG. 11, FIG. 12 and FIG. 13, the whole second locating block 22 is U-shaped, and has a lateral insertion slot 22a in an insertion fit with the mounting portion 212. The lateral insertion slot 22a has a through slot structure running through from top to bottom, and the second locating block 22 can be laterally mounted on the mounting portion 211 of the first locating block 21 through the lateral insertion slot 22a. In this way, the vertical center line of the second locating block 22 can coincide with the axis of the push rod 213 as much as possible, thereby increasing the contact area between the second locating block 22 and the thumb rest 131 of the syringe and ensuring the mounting stability of the second locating block 22.

It can be understood that when the second locating block 22 is laterally mounted on the mounting portion 211 of the first locating block 21, the direction of the opening of the lateral insertion slot 22a is the same as or opposite to the direction of the opening of the flange insertion slot 21a, which is not limited here. In this example, the direction of the opening of the lateral insertion slot 22a is preferably the same as the direction of the opening of the flange insertion slot 21a.

In a more specific example, as shown in FIG. 12 to FIG. 14, both the guide rail 212 and the recess 223 are U-shaped, which effectively improves the mounting stability of the second locating block 22.

In order to facilitate mounting and demounting of the second locating block 22, as shown in FIG. 13 to FIG. 15, an outer side wall of the second locating block 22 is formed with two hand-held members 225. The two hand-held members 225 are arranged symmetrical with respect to the lateral insertion slot 22a, and the hand-held member 225 is formed with a finger recess 225a that fits into a finger, so as to improve the operating comfort of the operator.

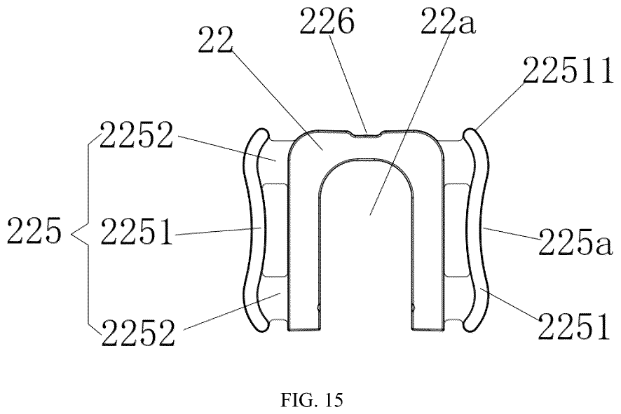

In a preferred example, as shown in FIG. 15, the hand-held member 225 includes a hand-held portion 2251 and two connecting arms 2252 arranged on the hand-held portion 2251 at an interval, the hand-held portion 2251 is connected to the second locating block 22 through the connecting arms 2252, and the finger recess 225a is located on a side of the hand-held portion 2251 away from the connecting arm 2252. Since the two connecting arms 2252 are arranged at an interval, a notch is formed. When the second locating block 22 and the hand-held member 225 are integrally formed, the notch can effectively reduce the influence of thermal expansion and contraction on the formed member, thereby improving the forming quality. In this example, the whole hand-held portion is 2251 wavy, which makes the hand-held portion 2251 more beautiful.

In actual operation, in order to prevent the second locating block 22 from sliding off the mounting portion 211 under the action of gravity when the opening of the lateral insertion slot 22a faces up, as shown in FIG. 12 and FIG. 13, an inner wall of the lateral insertion slot 22a is provided with two sets of first clamping members, and the two sets of first clamping members are arranged symmetrically with respect to the recess 223. Each set of first clamping members includes two first clamping members arranged symmetrically with respect to the axis of the push rod, and the first clamping members are preferably arranged close to the opening of the lateral insertion slot 22a. An outer wall of the mounting portion 211 is provided with second clamping members clamped with the first clamping members. One of each of the first clamping members and each of the second clamping members is a clamping protrusion 213, and the other is a clamping groove 224. After the second locating block 22 is mounted in position, the first clamping members on the inner wall of the lateral insertion slot 22a will be clamped with the second clamping members on the outer wall of the mounting portion 211, so as to limit the insertion and removal of the second locating block 22.

However, after the first clamping members are clamped with the second clamping members, it is difficult to remove the second locating block 22. In order to reduce the difficulty in removing the second locating block 22, as shown in FIG. 15, an end of the hand-held portion 2251 away from the opening of the lateral insertion slot 22a is a cantilever end, which is a press end. When the operator presses the press ends of the two hand-held portions 2251 at the same time, two opposite side walls of the lateral insertion slot 22a will rotate outward, so that the lateral insertion slot 22a opens, thereby reducing the difficulty in removing the second locating block 22.

The press end is provided with a press surface 22511, and the press surface 22511 is an anti-slip surface, so that when the operator presses the two press surfaces 22511, the two press ends can come close to each other. Thus, the two opposite side walls of the lateral insertion slot 22a can rotate outward and open, thereby reducing the difficulty of removing the second locating block 22.

In order to further reduce the difficulty in opening the lateral insertion slot 22a, as shown in FIG. 15, a side wall of the second locating block 22 is convenient for a thinning slot 226 for facilitating the opening of the lateral insertion slot 22a. The thinning slot 226 is located on the side wall of the second locating block 22 away from the opening of the lateral insertion slot 22a.

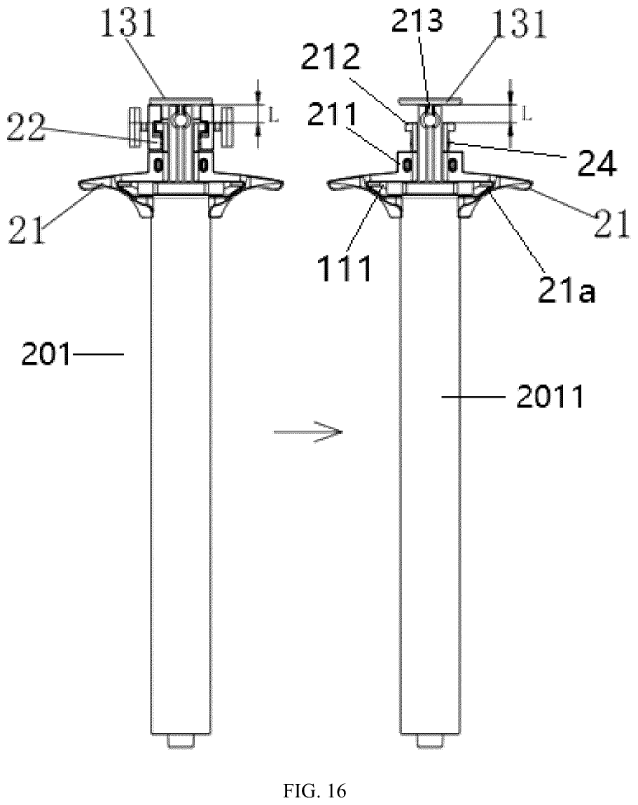

When microinjection is needed, as shown in FIG. 16, first, the micro assistor 2 is laterally mounted on the flange 111 of the injection barrel 2011, so that the micro assistor 202 is mounted to the prefilled syringe 201. Then, the thumb rest 131 of the syringe is pressed such that the thumb rest 131 of the syringe contacts the second locating block 22, thereby determining the injection start position. At this time, the distance between the top end of the second locating block 22 and the top end of the mounting portion 211 is L. Next, the second locating block 22 is removed, thereby completing the preparations before injection.

The operator presses the thumb rest 131 of the syringe to start injection. When the thumb rest 131 of the syringe comes into contact with the top end of the mounting portion 211, it indicates that the thumb rest 131 of the syringe has reached the injection end position, and the moving stroke is the distance L. Since the accuracy of the injection dose is affected only by the machining error of the inner diameter of the injection barrel 2011 and the assembling error of the two locating blocks, the accuracy of the injection dose is high, which is beneficial to meet the requirements of high-accuracy microinjection.

When the cushion portion 221 and the insert portion 222 of the stopping member 5 have the same thickness, the control accuracy can be improved as compared with the case where the cushion portion 221 and the insert portion 222 have different thicknesses. This is because when the cushion portion 221 and the insert portion 222 have different thicknesses, the operator may mistakenly mount the stopping member. If the cushion portion 221 and the insert portion 222 have the same thickness, errors can be avoided to the greatest extent, thereby further improving the control accuracy of the distance a.

It should be understood that although the disclosure has been described in detail through specific embodiments, these descriptions are not intended to limit the scope of the disclosure, and any changes in form and details made on this basis that are not beyond the scope of the claims shall fall into the scope of the disclosure.

1. An injection device without changing an original structure of a syringe, comprising a barrel (1) and a push rod (2) slidably connected in the barrel (1); wherein the push rod (2) or the barrel (1) is connected with a stopping member (5), and the stopping member (5) is capable of controlling a displacement distance of the push rod (2) in the barrel (1), thereby controlling an injection dose.

2. The injection device according to claim 1, wherein the stopping member (5) is provided with a first stopping portion (51) and a second stopping portion (52) at different positions; a distance between the first stopping portion (51) and the second stopping portion (52) is a fixed distance a; the push rod (2) is moved, and when the push rod (2) is stopped by the first stopping portion (51), the injection device is in an injection start position; and when the push rod (2) is stopped by the second stopping portion (52), the injection ends.

3. The injection device according to claim 2, wherein the stopping member (5) is located on the push rod (2) outside the barrel.

4. The injection device according to claim 3, wherein the barrel is provided with a flange (8), and one end of the push rod is provided with a thumb rest (4); when the thumb rest (4) or the flange (8) abuts against the first stopping portion (51), the injection device is in the injection start position; and when the thumb rest (4) or the flange (8) abuts against the second stopping portion (52), the injection ends.

5. The injection device according to claim 4, wherein the push rod (2) is clamped by the stopping member (5); and the other end of the push rod (2) is provided with a piston (3), and a gap is left between the piston (3) and a bottom of the barrel (1).

6. The injection device according to claim 5, wherein one end of the stopping member (5) abuts against the thumb rest (4); the other end of the stopping member (5) extends outward along a radial direction of the push rod (2) to form a first protrusion (501), and an end of the first protrusion (501) away from the stopping member (5) extends toward the barrel (1) to form a second protrusion (502); an end surface of the second protrusion (502) facing the barrel (1) is the first stopping portion (51); an end surface of the first protrusion (501) facing the barrel (1) is the second stopping portion (52); when the flange (8) abuts against the first stopping portion (51), the injection device is in the injection start position; and when the flange (8) abuts against the second stopping portion (52), the injection ends.

7. The injection device according to claim 5, wherein the stopping member (5) comprises a first locating block (503) and a second locating block (504), and the first locating block (503) and the second locating block (504) are connected and separated detachably.

8. The injection device according to claim 7, wherein the first locating block (503) is clamped on the flange (8), and a top of the first locating block (503) is connected with the second locating block (504); an end surface of the second locating block (504) facing the thumb rest (4) is the first stopping portion (51), and an end surface of a top of the first locating block (503) facing the thumb rest (4) is the second stopping portion (52); when the thumb rest (4) abuts against the first stopping portion (51), the injection device is in the injection start position; and when the thumb rest (4) abuts against the second stopping portion (52), the injection ends.

9. The injection device according to claim 8, wherein the top of the first locating block (503) extends upward to form a third protrusion (505), and an end surface of at least one side of the third protrusion (505) is provided with a guide groove (11); an end surface of the second locating block (504) facing the first locating block (503) is provided with a recess (12), and an inner wall of the recess (12) is provided with guide protrusions (13) in equal number and corresponding position to the guide grooves (11); and the guide protrusion (13) is slidably connected to the guide groove (11).

10. The injection device according to claim 9, wherein a bottom of the recess (12) is provided with a through slot (14) for the push rod (2) to run through.

11. The injection device without changing an original structure of a syringe according to claim 10, wherein an outer side end surface of the second locating block (504) is provided with an anti-slip rib (15).

12. The injection device according to claim 9, wherein the third protrusion (505) is a mounting portion (211) of the second locating block (504); the second locating block (504) comprises a cushion portion (221) and an insert portion (222), and the recess (12) is located between the cushion portion (221) and the insert portion (222); and the second locating block (504) is detachably mounted on the mounting portion (211) of the first locating block (503) through the insert portion (222).

13. The injection device according to 12, wherein an end of the mounting portion (211) close to the thumb rest (4) protrudes to form a guide rail (212) fitted with the recess (12), and a guide slot (24) is arranged below the guide rail (212); and when the second locating block (504) is mounted on the mounting portion (211) of the first locating block (503), the insert portion (222) is located in the guide slot (24).

14. The injection device according to claim 13, wherein a thickness of the cushion portion (221) and/or the insert portion (222) is not greater than a width of the guide slot (24); and the cushion portion (221) and the insert portion (222) are interchangeable.

15. The injection device according to claim 14, wherein the cushion portion (221) and the insert portion (222) have the same thickness.

16. The injection device according to claim 15, wherein the whole second locating block (504) is U-shaped, and has a lateral insertion slot (22a) in an insertion fit with the push rod (2); and the first locating block (503) is provided with a push rod dodging slot (211a) in an insertion fit with the push rod (2), and also provided with a flange insertion slot (21a) matched with the flange (8).

17. The injection device according to 16, wherein an outer side wall of the second locating block (504) is provided with two hand-held members (225), and the two hand-held members (225) are arranged symmetrically; and the hand-held member (225) comprises a hand-held portion (2251) and two connecting arms (2252) arranged on the hand-held portion (2251) at an interval, and the hand-held portion (2251) is connected to the second locating block (504) through the connecting arms (2252).

18. The injection device according to claim 17, wherein the hand-held portion (2251) has a cantilever-shaped press end: when the two press ends are close to each other, two opposite side walls of the lateral insertion slot (22a) open outward; and the press end is provided with a press surface (22511), and the press surface (22511) is an anti-slip surface.

19. The injection device according to claim 18, wherein an inner wall of the lateral insertion slot (22a) is provided with first clamping members, and an outer wall of the third protrusion (505) is provided with second clamping members clamped with the first clamping members.

20. The injection device according to claim 19, wherein one of each of the first clamping members and each of the second clamping members is a clamping protrusion (213), and the other is a clamping groove (224).
